(19) United States
(12) Patent Application Publication
(10) Pub. No.: US 2007/0239095 A1
(43) Pub. Date: Oct. 11, 2007

(54) Shoulder traction and rotation sleeve with silicone impregnated webbing

(75) Inventors: Craig J. Koloske, Tracy, CA (US); Chad M. Sindel, Pleasant Hill, CA (US); John Petlansky, Tracy, CA (US); Paul Wiese, Livermore, CA (US)

Correspondence Address: DICKSTEIN SHAPIRO LLP, 1825 EYE STREET NW, Washington, DC, 20006-5403, US

(73) Assignee: Top Shelf Manufacturing, LLC

(21) Appl. No.: 11/723,513

(22) Filed: Mar. 20, 2007

Related U.S. Application Data
(60) Provisional application No. 60/783,865, filed on Mar. 21, 2006.

(51) Int. Cl. A61F 5/00

(52) U.S. Cl. 602/36; 602/32

(57) ABSTRACT

A shoulder traction and rotation sleeve including a soft traction boot designed to cradle the arm, forearm and wrist in a proper position during arthroscopic shoulder surgery. The traction boot may be formed of a flat foam or a spandex material. The sleeve further includes at least one silicon impregnated webbing strip on the inside of the traction boot to maintain engagement with the arm and prevent migration. The sleeve may further include a traction strap formed of a nylon webbing and a D-ring traction loop.

### CROSS REFERENCE TO RELATED APPLICATIONS

This application claims the benefit of U.S. Provisional Application No. 60/783,865, filed Mar. 21, 2006.

### FIELD OF THE INVENTION

The present invention relates to a shoulder traction and rotation sleeve that provides lateral traction and rotation of the arm, to improve visualization of the shoulder joint during arthroscopic and general repairs of torn tissue in the shoulder.

### BACKGROUND OF THE INVENTION

The rotator cuff is formed of four tendons that stabilize and move the shoulder. When a tear occurs in the rotator cuff of the shoulder, it is often necessary to reattach the torn tendon to the bone of the humeral head. Rotator cuff repair is generally difficult in that the torn cuff is first punctured by a punch and prethreaded suture anchor screws (soft tissue fasteners) are drilled into the head of the humerus bone, to allow the sutures to pass through the cuff using suture relay devices that pass the sutures through the tissue. After the suture strands are passed through the tissue, they are knotted and tied together to secure the reattached rotator cuff to the humerus head. These steps are conducted with the shoulder distracted distally, to provide maximum visual field of the shoulder joint and to allow the surgeon to conduct reattachment and retensioning of the torn ligaments, and to maneuver the suture and tissue with the small suture relay devices. In order to effectively perform surgery, and to provide access and a proper view to the surgeon, the should must be properly aligned and held in place during surgery. Traditionally, this as been done by a surgical assistant.

Shoulder traction sleeves are now available which alleviate the need for a surgical assistant in holding the shoulder in proper alignment during a surgery. However, these models of shoulder traction sleeves are made from an egg-crate type of foam in order to maintain engagement of the arm and prevent migration during surgery. This construction causes the sleeve to be thick and bulky. Accordingly, an improved device for holding the arm in the proper position during shoulder surgery is desired.

### SUMMARY OF THE INVENTION

The present invention provides a Shoulder Traction and Rotation sleeve (STaR sleeve) that comprises a sterile traction boot designed to gently cradle the arm, forearm and wrist during distal distraction in any desired position of abduction. The STaR sleeve includes a lateral traction and rotation strap that provides lateral traction to improve visualization in the shoulder joint. When a rotator cuff repair is performed in the lateral position, the lateral strap can be used to hold the arm in internal or external rotation, obviating the need for additional surgical assistance to perform this task. The STaR sleeve of the present invention further includes strips of silicon impregnated webbing on the inside of the sleeve to prevent migration during surgery.

Other features and advantages of the present invention will become apparent from the following description of the invention which refers to the accompanying drawings.

### BRIEF DESCRIPTION OF THE DRAWINGS

FIG. 1 illustrates a side view of a STaR sleeve according to an embodiment of the present invention.

FIG. 2 illustrates a top view of the STaR sleeve of FIG. 1.

FIG. 3 illustrates a view of the STaR sleeve of FIG. 1 in use.

### DETAILED DESCRIPTION OF THE INVENTION

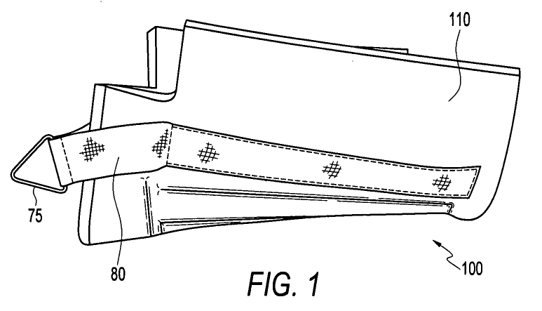

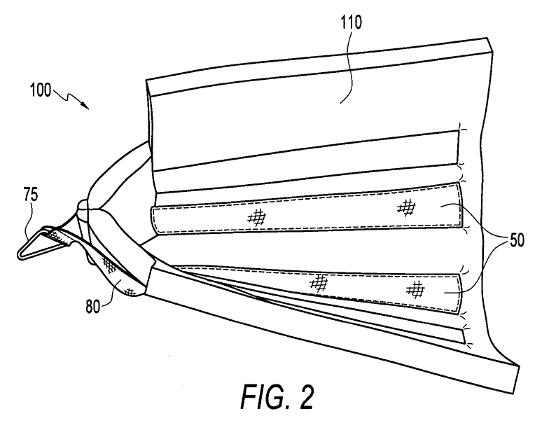

Referring now to the drawings, where like elements are designated by like reference numerals, FIGS. 1-3 illustrate an exemplary embodiment of a Shoulder Traction and Rotation sleeve (STaR sleeve) 100 according to an embodiment of the present invention. The STaR sleeve 100 of FIG. 1 includes of a sterile, soft foam traction boot 110 designed to gently cradle the arm, forearm and wrist during distal distraction in any desired position of abduction. In other embodiments, the traction boot 110 may be formed of a spandex material or any other suitable material known in the art. At least one strip of silicone impregnated webbing 50 is provided on the inside of the STaR sleeve 100. Preferably, two strips of silicone impregnated webbing 50 are attached, by sewing, for example, on the inside of the STaR sleeve 100 to serve as a “grip tape” that holds the arm and wrist secure and prevents migration of the arm during surgery. The sleeve may be optionally wrapped with an elastic band (not shown) for additional circumferential hold.

Previous types of sleeves were formed of a complicated egg-crate type foam in order to maintain engagement with the arm, which caused the sleeves to be bulky. The traction boot 110 of the STaR sleeve 100 of the present invention, on the other hand, is formed of a flat foam or a spandex material, making it less bulky in surgery rooms and storage settings than previous sleeves. To maintain engagement with the arm during shoulder surgery, the STaR sleeve 100 of the present invention instead uses the two strips of silicone impregnated webbing 50 sewn into the inside of the sleeve rather than the shaped foam.

A lateral traction and rotation strap, formed of a nylon webbing 80 with a D-ring traction loop 75, is included with each STaR sleeve 100 provides lateral traction to improve visualization in the shoulder joint. The nylon webbing 80 is sewn along the entire length of the traction boot 110 to provide additional strength during tractioning. When a “mini-open” rotator cuff repair is performed in the lateral position, the lateral strap can be used to hold the arm in internal or external rotation, obviating the need for a surgical assistant to perform this task.

During surgery, the STaR sleeve 100 may be used with a 3-Point Shoulder Distraction System or a Modular Joint Distractor. The 3-Point Shoulder Distraction System provides safe, effective and easy positioning of the shoulder during all types of arthroscopic or open shoulder surgery performed in the lateral decubitus position. The 3-point shoulder traction with a lateral strap permits ideal shoulder positioning for improved access to the anterior glenohumeral joint. During arthroscopy, single point traction may be selected at any desired angle of abduction by transferring weight to the third traction cable.

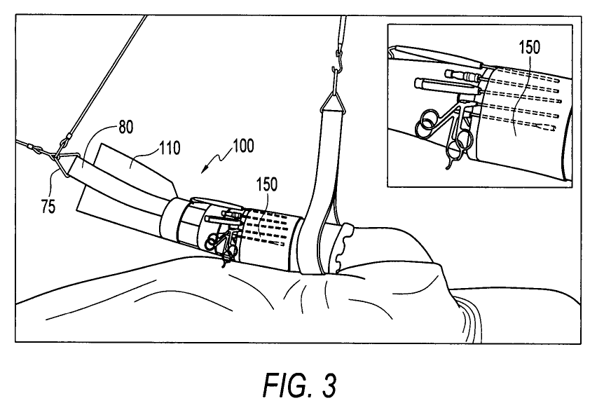

As shown in FIG. 3, a disposable, soft plastic sleeve 150 may be fastened around the STaR sleeve 100, if desired, to conveniently allow storing of arthroscopic instruments in the plastic sleeve 150, during surgery. This provides easier access to the instruments needed by the surgeon.

Although the present invention has been described in connection with preferred embodiments, many modifications and variations will become apparent to those skilled in the art. While preferred embodiments of the invention have been described and illustrated above, it should be understood that these are exemplary of the invention and are not to be considered as limiting. Additions, deletions, substitutions, and other modifications can be made without departing from the spirit or scope of the present invention.

What is claimed as new and desired to be protected by Letters Patent of the United States is:

1. A shoulder traction and rotation sleeve comprising:
a traction boot adapted to cradle an arm, forearm and wrist in a desired position of shoulder abduction;
at least one strip of silicon impregnated webbing provided on an inside surface of the traction boot; and
a strap provided on the outside of the traction boot adapted for providing traction.

2. The shoulder traction and rotation sleeve of claim 1, wherein there are at least two strips of silicon impregnated webbing provided on the inside of the traction boot.

3. The shoulder traction and rotation sleeve of claim 1, wherein the silicon impregnated webbing is adapted to prevent migration of the traction boot during a surgery.

4. The shoulder traction and rotation sleeve of claim 1, wherein the traction boot is formed of foam.

5. The shoulder traction and rotation sleeve of claim 4, wherein the foam is a flat foam.

6. The shoulder traction and rotation sleeve of claim 1, wherein the traction boot is formed of a spandex material.

7. The shoulder traction and rotation sleeve of claim 1, wherein the strap further includes a D-ring traction loop.

8. The shoulder traction and rotation sleeve of claim 1, wherein the strap is formed of nylon webbing.

9. The shoulder traction and rotation sleeve of claim 1, wherein the strap is sewn along an entire length of the traction boot.

10. The shoulder traction and rotation sleeve of claim 1, further comprising an elastic band provided around the traction boot, the elastic band being adapted for circumferential hold of the traction boot.

11. The shoulder traction and rotation sleeve of claim 1, further comprising a plastic sleeve provided on the outside of the traction boot, the plastic sleeve being adapted for holding surgical instruments during surgery.
